(19) United States
(12) Patent Application Publication
(10) Pub. No.: US 2015/0369784 A1
(43) Pub. Date: Dec. 24, 2015

(54) DEVICE FOR MEASURING RESIDUAL OIL

(71) Applicant: BEKO TECHNOLOGIES GMBH, Neuss (DE)

(72) Inventor: Martin FRIEDRICH, Loffingen (DE)

(21) Appl. No.: 14/764,844

(22) PCT Filed: Jan. 20, 2014
(86) PCT No.: PCT/EP2014/051041

§ 371 Date: Jul. 30, 2015

(30) Foreign Application Priority Data
Jan. 30, 2013 (DE) 10 2013 100 950.6

(51) Int. Cl. G01N 33/00

(52) U.S. Cl. CPC G01N 33/0032; G01N 33/0047

(57) ABSTRACT

The invention relates to a measuring device for detecting amounts of hydrocarbon in gases, and comprising—a first sensor (22) for determining the amount of hydrocarbon in a first measurement gas flow (38) and for producing a corresponding first measurement result,—a second sensor (24) for determining the amount of hydrocarbon in a second measurement gas flow (39) and for producing a corresponding second measurement result, and—an evaluation unit for evaluating the measurement results of the two sensors (22, 24),—the first sensor (22) being a metal oxide semiconductor gas sensor and carrying out measurements continuously, and—the second sensor (24) being a photoionisation sensor and carrying out measurements intermittently. (FIG. 1) The invention also relates to a method for recording the amount of hydrocarbon in a gas flow.

### TECHNICAL FIELD

The present invention relates to a measuring device and a method for detecting the hydrocarbon content in gases.

### BACKGROUND

Such measuring devices are known with various sensor technologies and serve for detecting the content of oil, hydrocarbons and oxidizable gases in, for example, air or compressed air.

For example, electrically heatable metal oxide semiconductor gas sensors with semiconductor oxide materials are used frequently, which in the heated state change their electrical resistance depending on the amount of hydrocarbons contained in the air. The most important advantages of metal oxide semiconductor gas sensors include the very high sensitivity, and thus the possibility of being able to measure even the most minute hydrocarbon contents down to the ppt range. They have a very long operating life, a very good long-term stability, and the acquisition costs are rather low.

However, metal oxide semiconductor gas sensors are disadvantageous in that they have an exponential characteristic curve, which is why their offset point is difficult to determine. The measurement results are relatively hard to reproduce, and the sensors have high cross sensitivities to water vapor and inorganic gases. The response times to the final value are high and the recovery times in the case of a calibration with zero air until the zero line has been reached are relatively long.

Another method is the detection of the hydrocarbon concentration by means of photoionization. In the process, the hydrocarbons are irradiated with ultraviolet light. In this case, the amount of energy of the light has to be sufficiently high for electrons to be forced out of the hydrocarbon molecule. Their number can be measured electronically. Photoionization sensors have a good long-term stability, a relatively low cross sensitivity to water vapor and inorganic gases. The response times to the final value are short, as are the recovery times, in the case of a calibration with zero air, until the zero line has been reached. The characteristic curve is linear, so that a high level of reproducibility is provided.

However, what is disadvantageous in sensors of this kind is their low level of sensitivity, which is relevant particularly in the low-concentration range. Also, the state of ageing cannot be determined reliably without a test gas; however, the operating life of about one year is rather short anyway. The measuring accuracy decreases due to wear, which is why the maintenance costs are high. Moreover, the acquisition costs of photoionization sensors are relatively high.

The measured values generated by means of photoionization sensors permit only indirect conclusions to be drawn with regard to the measured substance quantity, because the measured values are also dependent on the molecular build of the compound and vary to a rather considerable extent, even in the case of identical molecular formulas. If, however, the compound to be measured is constant, known and, if possible, uniform, the concentration of the hydrocarbon content can be measured relatively reliably. However, measuring accuracy decreases as the concentration of hydrocarbons drops. In particular, the influence of the moisture content of the air increases in the process. As the hydrocarbon content decreases, the influence of air humidity becomes increasingly large; measurements of hydrocarbon content in the lower mg/m³ range and, in particular, in the μg/m³ range cannot be carried out with sufficient accuracy.

Different applications of compressed air demand different threshold values for the oil content. Oil contents consist of drop-like oil aerosols and of oil vapors. Oil aerosols and oil vapors can be partially or largely eliminated from the compressed-air flow by various methods.

### BRIEF SUMMARY

The disclosure provides a measuring device for detecting the content of oil, hydrocarbons and oxidizable gases in gases that reliably measures even the lowest permanent concentrations. Possible measurement errors are supposed to be easy to determine and correct. The disclosure further provides a method for detecting of the content of oil, hydrocarbon contents and oxidizable gases in gases that is improved over the prior art.

More specifically, the disclosure provides a measuring device for detecting hydrocarbon contents in gases, comprising
a first sensor for determining the hydrocarbon content in a measuring gas flow and for producing a corresponding first measurement result,
a second sensor for determining the hydrocarbon content in the measuring gas flow and for producing a corresponding second measurement result,
an evaluation unit for evaluating the measurement results of the two sensors,
a catalyst unit (34) for producing a catalyst gas flow (36),
   wherein
the first sensor is configured as a metal oxide semiconductor gas sensor and continuously carries out measurements,
the second sensor is configured as a photoionization sensor and discontinuously carries out measurements,
the catalyst gas flow (36) can be fed to the second sensor (22).

Furthermore, a method is herein provided for detecting the hydrocarbon content in a gas flow, which is characterized by the method steps:
continuously feeding a measuring gas flow to a first sensor configured as a metal oxide semiconductor gas sensor,
determining the hydrocarbon content in the measuring gas flow and producing a first measurement result by means of the first sensor,
discontinuously feeding the measuring gas flow to a second sensor (24) configured as a photoionization sensor,
determining the hydrocarbon content in the measuring gas flow and producing a second measurement result by means of the second sensor,
evaluating the measurement results of the two sensors,
producing a catalyst gas flow (36), wherein the catalyst gas flow (36) is fed to the second sensor (24) when the second sensor (24) is turned off or not used.

For the first time, the measuring device according to the invention combines the two different sensors which, usually, mutually exclude each other in a measuring device. So far, it was considered superfluous to use both sensors in a single measuring device.

As an advantage over the photoionization sensor, PID sensor in short, the metal oxide semiconductor gas sensor, MOX sensor in short, offers a long life expectancy without any maintenance as well as low acquisition costs. Theoretically, several years of use without any maintenance and recalibration can be realized for an oil vapor measuring device.

With respect to cross sensitivity, reproducibility and long-term stability, the PID sensor is the more accurate sensor; it permits more precise measurements and has a lower cross sensitivity.

One aspect of the invention is fact that the two sensors are used differently. The MOX sensor is permanently in operation and carries out measurements continuously. In contrast, the PID sensor only measures in cyclic intervals, for instance once daily, in addition to and in parallel with the MOX sensor. The determined measured values of the PID sensor then serve, for example, for correcting a slope or the offset of the MOX sensor. In this case, the measurement of the PID sensor takes place directly after a zero air calibration that has been previously carried out preferably automatically.

A considerably longer operating life of this otherwise rather short-lived sensor is achieved by the discontinuous additional activation of the PID sensor.

Both sensors have a relatively high cross sensitivity to water. In the case of MOX sensors, this cross sensitivity results in a shift of the operating point on its exponential characteristic curve, and thus to an offset and/or gain error.

In contrast, two effects are known in the case of the PID sensor; one is the so-called water quenching effect, which leads to a slope error. In addition, leak currents may occur due to depositions or contaminations on the electrode stack, which lead to a moisture-dependent offset error. According to the invention, a drying element, e.g. a membrane, which significantly reduces the water content, can therefore be provided on the gas inlet of the measuring device. Known membrane dryers can be used for this purpose, which dry the air itself and use expanded flushing air for the drying process. The use of adsorption dryers is also possible.

The use of a drying element in the original gas flow (measuring gas flow), for example of a membrane dryer, has advantages both for the use of the PID sensor as well as for the use of the MOX sensor, which is furthermore advantageous over the art that usually does not combine these two sensor types.

In principle, the use of an adsorption dryer is also possible. What is essential is that the drying element has a high drying capacity, which is often not sufficiently provided for in adsorption dryers.

Furthermore, a catalyst unit is provided according to the invention, which enables an offset stabilization of the PID sensor with zero air. According to the invention, the PID sensor is flushed with a catalyst gas flow (catalyzed measuring gas) whenever it is not used and turned off. Thus, the PID sensor is always kept clean, and the stability and service life is increased. Only just before the PID sensor is operated in parallel with the MOX sensor, voltage is applied to the PID sensor and its lamp is turned on. After a sufficient stabilization time, an automatic zero adjustment takes place. After this adjustment, the original gas flow is divided into a first and a second measuring gas flow, and the second measuring gas flow is fed to the PID sensor. The PID sensor then measures this second measuring gas flow in parallel with the MOX sensor, which measures the first measuring gas flow.

With the measurement result of the PID sensor it is possible to compensate the offset of the MOX sensor or also the gain error of the MOX sensor or to calibrate the MOX sensor.

The use of the catalyst unit not only permits a mere offset stabilization of the PID sensor but, according to the invention, also the compensation of the cross sensitivity of the MOX sensor and the determination of the offset point of the MOX sensor on its characteristic curve.

The compensation of the cross sensitivity of the MOX sensor by means of the PID sensor is based on the different ways the sensors operate. The MOX sensor has an exponential characteristic curve which is calibrated during the manufacture of the sensor and is stored in the device. In contrast, the PID operates almost linearly and is calibrated with zero air. The PID sensor is thus able to measure very accurately the zero value in a measurement process around Class 1 (ISO 8573: oil vapor contents below 0.01 mg per m³ gas).

The compensation of the cross sensitivity of the MOX is carried out by calibrated points of the measured values of the PID sensor (also the points measured earlier) being mapped on the exponential curve of the MOX sensor if the measured values are worse than Class 1. The current measured value is used in the process as a reference quantity for a statistical probability calculation of all previously determined calibration points; the improbable points are removed from the collection of measured values. With the mean value of all other, i.e. probable, calibration points, the new slope is determined and the system slope is slowly adjusted via a filter.

In contrast, for measured values better than Class 1, the offset value is corrected and the operating point is determined on the exponential characteristic curve of the MOX sensor for the offset value. By means of a mathematical algorithm, it is possible, even with the MOX sensor, which actually has much too large a cross sensitivity for measurement in Class 1, to measure down to Class 1 and better.

The measuring device has an evaluation unit for evaluating the measurement results. In a first embodiment, the two sensors can be connected to a single evaluation unit; however, it is also conceivable that each sensor is allocated its own evaluation unit. The evaluation unit has a processor that carries out the necessary calculations. The individual gas flows or the sum of all gas flows can be measured by the sensor or sensors, or be evaluated by the evaluation unit.

Moreover, a display unit is provided which displays the measurement results of the sensors or values calculated by the evaluation unit and/or other information. Advantageously, the display unit can also be configured as an input unit in the form of a touch screen.

By using the two sensors, it is also accomplished that the measuring device can continue to be operated in an emergency mode even if one of the two sensors fails. The evaluation unit is also capable of turning off one of the two defective sensors or gas paths autonomously, so that the measuring device continues to be operable. Advantageously, the evaluation unit outputs corresponding information via the display unit so that the measuring device can be repaired prior to a failure of the second gas path or sensor and the accompanying total failure. Another essential advantage results from the fact that, if necessary, the corresponding repair can be carried out during a later break in operation that is coming up anyway.

Finally, the measuring device is capable, with a corresponding error analysis program, to check all components in the gas path autonomously. The flushing of the sensors with a reference gas can also be initialized autonomously by the measuring device in regular cycles or because of a deviating or suspicious measurement result.

Further, the measuring device according to the invention can be configured in such a way that a reference gas flow, for example from a storage bottle, can be applied to each of the two sensors. In addition to the unchanged measuring gas and the catalyzed measuring gas, a reference gas flow is thus also used regularly, for example for re-determining the signal strength coming from the sensor. The calibration gas (e.g. isobutene) has a defined hydrocarbon content but no moisture or only a very low moisture. It is thus possible, according to the invention, to reliably compensate the change of the signal strength and measurement sensitivity due to ageing and contamination of the measuring device. The calibration measurement can take place automatically at regular intervals; however, it can also be triggered at any time by the user. In particular, it can be used by the error analysis program within the context of error analysis. The data determined within the context of the calibration measurement are stored and can be retrieved and used by the evaluation unit at any time.

According to the invention, a reference gas can be applied simultaneously to the PID sensor and the MOX sensor. This function can be used for a cyclic autocalibration of the device but also for a recalibration within the context of servicing. The reference gas typically has a concentration in the upper measurement range, e.g. 500 ppb. After the start of the feed of the reference gas to the two sensors, the gain value of both sensors is calibrated after an appropriate stabilization time.

In the case of a parallel feed of the reference gas to both sensors, no measurement of hydrocarbon contents can take place during the gain calibration. According to the invention, however, a separate valve can be provided for both sensors, which permits the feed to only one of the two sensors, whereby the gain calibration can be carried out separately and the respective other sensor can continue to be used for the continuous measurement.

The measuring device can be produced particularly inexpensively if technical units are combined with each other in the form of building blocks. This may relate to, for example, valves, throttles, catalysts and sensors. The corresponding elements and components are made of metal, for example.

The PID sensor has a sensor unit with a sampling probe; preferably, both sensors use a sampling probe jointly. In this case, the sensor unit is connected to the evaluation unit via a signal cable or wirelessly. Preferably, the sampling probe can be mounted centrally in a riser from above so that it is able to withdraw gas from the center of the gas flow to be monitored. The sensor unit has defined flow resistors that provide for a constant pressure and a constant volume flow of the individual measuring gases and are formed, for example, by a throttle with a defined bore or from a sintered metal. They are particularly low-maintenance and easy to clean. Further, an alarm function is provided which informs the user visually or acoustically in case of too low or too high a pressure of the gas flows.

The flow rate of the different gas flows can be influenced with corresponding throttles, valves or flow reducers. They are preferably replaceable and, in a particularly advantageous embodiment, controllable in order to be able, on the one hand, to adjust the flow rate to the sensors and, on the other hand, to reliably ensure the desired mixing ratios of the gas flows to be mixed.

Due to the fact that the valves in the gas path are individually switchable, it is also possible to switch the reference air and the measuring air to the sensor at the same time and to dilute the measuring gas. Thus, the measurement range can be expanded upwards if the measuring air is extremely contaminated.

Common oxidation catalysts can be used as catalyst gas units; however, other devices or methods for providing gases with the desired properties are also conceivable. Platinized quartz wool, for example, which can be inserted in a container provided for this purpose without any trouble, serves as an oxidation catalyst. The use of activated carbon is also conceivable. In a particularly advantageous variant, the reference gas generator is integrated into the measuring device, so that the various fluid and gas feeds only have to be connected on-site.

The measuring device thus has all connections for the corresponding gas pipes and also the electrical connection, so that it can be installed flexibly at any location on-site. In particular the division of the measuring device according to the invention into the sensor unit with sensors, e.g. the sampling probe in the case of the photoionization principle, and the evaluation unit with an operating panel (display), expands the possibilities of an on-site installation that is flexible with regard to its location. The evaluation unit with the operating panel is of a small build and can be installed almost anywhere, preferably at an easily accessibly position, whereas the slightly larger sensor unit can be disposed at a separate location from the evaluation unit at the measuring gas withdrawal point. However, the combination of the two components is also conceivable and advantageous. In that case, the device is compact and inexpensive, and because the oil vapor is present in its gaseous phase, a tube or a pipe can also be used for the feed. Moreover, the combined unit is more easily accessible for maintenance purposes.

Preferably, the measuring device according to the invention can be used with an oil-free compressing compressor for producing compressed air or compressed gas; however, the use with an oil-lubricated compressor is also conceivable if a corresponding catalyst is installed downstream from it. Preferably, a bypass is provided for maintenance work. In principle, however, the measuring device is also suitable for other areas of use, such as compressed-gas bottles.

### BRIEF DESCRIPTION OF THE DRAWINGS

The invention is explained in more detail below with reference to the attached Figures. In this case, the Figures merely show an advantageous embodiment in a greatly simplified schematic representation; under no circumstances is the invention to be limited thereto. In the drawings:

FIG. 1: shows a schematic diagram of the gas paths of the measuring device,

FIG. 2: shows a schematic diagram of the measurement cycle of the measuring device,

FIG. 3: shows a characteristic curve of the MOX sensor for explaining the compensation of the cross sensitivity,

### DETAILED DESCRIPTION

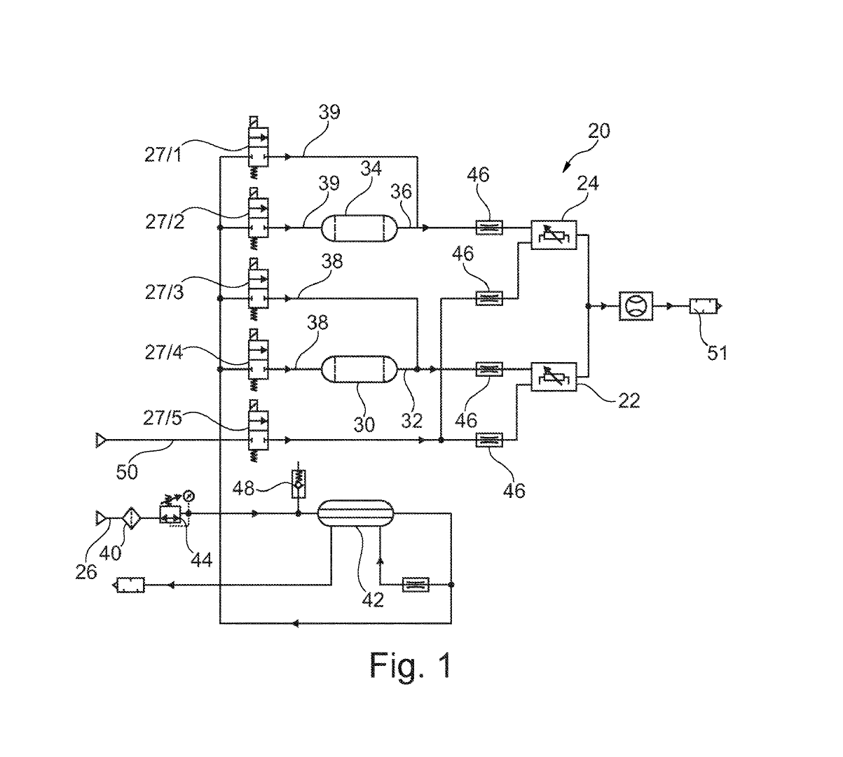

FIG. 1 shows a schematic diagram of the gas paths of the measuring device 20. It has two sensors, a metal oxide semiconductor gas sensor (hereinafter referred to as MOX sensor) as the first sensor 22, and a photoionization sensor (hereinafter referred to as PID sensor) as the second sensor 24.

An original gas flow 26 is divided into a first measuring gas flow 38 and a second measuring gas flow 39 via gas pipes and by means of valves 27.

In the exemplary embodiment shown, a catalyst unit 34, which generates a catalyst gas flow 36, is connected upstream from the second sensor 24. Analogously, a second catalyst unit 30, which generates a second catalyst gas flow 32, is connected upstream from the first sensor 22.

A filtering element 40 filtrates and a drying element 42 dries the original gas flow 26 and thus the two measuring gas flows 38, 39. The drying element 42 is preferably configured as a membrane dryer.

Moreover, a pressure regulator 44 and a safety valve 48 are provided in the original gas flow 26. Throttles 46, preferably expansion throttles, are connected upstream from the sensors 22, 24.

A first valve 27-1 switches the second measuring gas flow 39 and a second valve 27-2 switches the catalyst gas flow 36 to the second sensor 24. A third valve 27-3 switches the first measuring gas flow 38 and a fourth valve 27-4 switches the second catalyst gas flow 32 to the first sensor 22.

A reference gas flow 50 can be fed to the two sensors 22, 24 via a fifth valve 27-5. It preferably originates from an externally connected gas bottle, with the gas having a known hydrocarbon concentration, for example in the range from 300-1000 ppb, preferably 500 ppb.

Optionally, the measuring device has a sound absorber 51.

Thus, the second sensor 24 can be alternately supplied with the second measuring gas flow 39 or the catalyst gas flow 36 via the first valve 27-1 and via the second valve 27-2. Analogously, the first sensor 22 can be supplied with the first measuring gas flow 38 or the second catalyst gas flow 32 via the third valve 27-3 and the second valve 27-4. A reference gas flow 50 can be fed to both sensors via the fifth valve 27-5.

Particles are removed from original gas flow 26 by the filter element 40, and the original gas flow is adjusted to, for example, 3.8 bar by means of the pressure regulator 44. The original gas flow is then dried by means of the drying element 42, wherein the hydrocarbon content is not changed. Thus, a dried original gas flow 26 with a dew point of about minus 70° C. and an unchanged hydrocarbon content is available at the outlet of the dryer element. Both sensors 22, 24 can be operated with this dried original gas flow 26.

During operation, the first measuring gas flow 38 (i.e. the dried original gas flow 26) is continuously applied to the first sensor 22, the MOX sensor. The first valve 27-1 is closed, the second valve 27-2 is open, so that the sensor 24 is permanently flushed with the catalyst gas flow 36. In this case, the second sensor 24 is at first turned off. The third valve 27-3 is open, the fourth valve 27-4 and the fifth valve 27-5 are closed.

For the reference measurement with the second sensor 24, that is first turned on. After a sufficient stabilization time, i.e. a constant base line, the offset value of the second sensor 24 is recorded without any further switching of valves. Since the second valve 27-2 was already open, the second sensor 24 was flushed with the catalyst gas flow 36, i.e. zero air, and is free from hydrocarbons.

After the zero point has been recorded, the second valve 27-2 closes, and the first valve 27-1 is opened. The second sensor 24, the PID sensor, is now operated with the second measuring gas flow 39 and works in parallel with the first sensor 22.

After the second sensor 24 has determined measured values, the following decisions are made:

If the measured value is below a certain value (which typically corresponds to Class 1, approx. 5 ppb), the offset point of the first sensor 22 is corrected by means of an algorithm. The algorithm takes into account the exponential characteristic curve of the first sensor 22.

If the measured value is above Class 1, the slope of the characteristic curve of the first sensor 22 is corrected by means of an algorithm. This algorithm also takes into account the exponential characteristic curve of the first sensor.

After this reference measurement of the second sensor 24, that is switched back to zero air, i.e. the catalyst gas flow 36, and the operating voltage is turned off.

A zero point calibration can be carried out in the first sensor 22 by means of the fourth valve 27-4. For this purpose, the third valve 27-3 closes and the fourth valve 27-4 opens. Thus, the second catalyst gas flow 32, i.e. zero air, is applied to the first sensor 22. After a sufficient stabilization time, its offset can be calibrated.

The reference gas flow 50 can be applied to both sensors 22, 24 at the same time by means of the fifth valve 27-5. This can take place cyclically, within the context of an autocalibration of the device, but a recalibration within the context of a service procedure is also possible.

A gain calibration can be carried out by means of the reference gas flow 50. For this purpose, the first valves 27-1 to 27-4 are closed and only the fifth valve 27-5 is open. After an appropriate stabilization time, the gain value of the two sensors 22, 24 can be determined.

According to the invention, another valve which enables a separate gain calibration of the two sensors 22, 24 may be provided according to the invention.

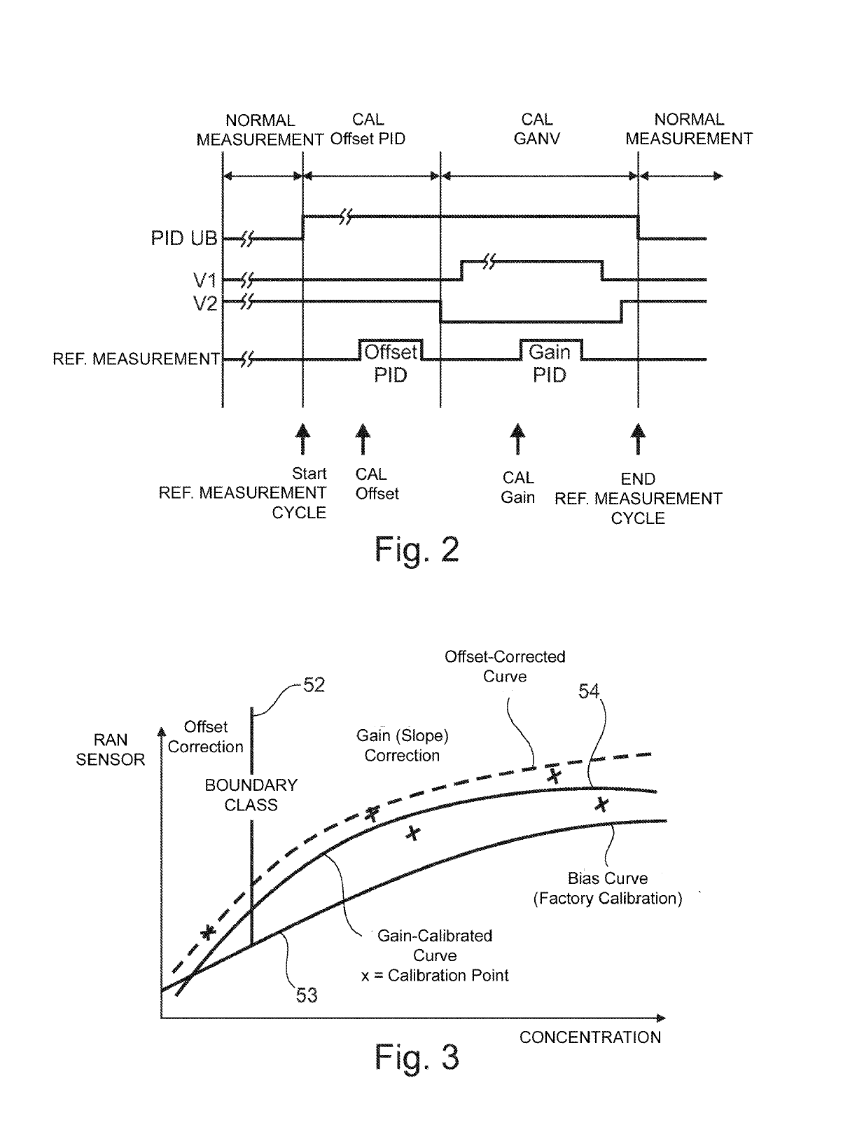

FIG. 2 illustrates the sequence in time of the measuring process described above.

FIG. 3 illustrates the calculation for the compensation of the cross sensitivity of the first sensor 22 based on the measurement results of the second sensor 24.

A class boundary 52 divides the curve into a range for offset correction and a range for gain (slope) correction. Apart from using the class boundary 52, other algorithms are also possible in order to modify offset and gain or even the exponential characteristic curve.

If the measured values measured by the second sensor 24 are worse than Class 1, i.e., if the concentration is higher, the current measured value of the second sensor is used as a reference quantity for a statistical probability calculation of all previously determined calibration points or measured values. The most improbable measured values are then removed from the collection of the measured values, and, using the mean value of the other, more probable, measured values, a new slope and thus a curve 54 corrected in relation to a basic characteristic curve 53 is determined and the system slope is slowly adjusted by means of a filter.

If the measured values are better than Class 1, an offset calibration is carried out and the operating point on the exponential characteristic curve of the first sensor 22 is determined for the offset value. Thus, it is possible with the first sensor 22, which actually has much too large a cross sensitivity, to measure down to Class 1 and obtain reliable measurement results.

The invention is not limited to the above-described exemplary embodiment; that serves only for description and is not to be understood as limiting.

1. A measuring device for detecting hydrocarbon contents in gases, comprising
a first sensor for determining the hydrocarbon content in a measuring gas flow and for producing a corresponding first measurement result,
a second sensor for determining the hydrocarbon content in a second measuring gas flow and for producing a corresponding second measurement result,
an evaluation unit for evaluating the measurement results of the two sensors,
a catalyst unit for producing a catalyst gas flow,
wherein
the first sensor is configured as a metal oxide semiconductor gas sensor and continuously carries out measurements,
the second sensor is configured as a photoionization sensor and discontinuously carries out measurements,
the catalyst gas flow can be fed to the second sensor.

2. The measuring device according to claim 1, wherein a second catalyst unit for the generation of a second catalyst gas flow, which can be fed to the first sensor, is provided.

3. The measuring device according to claim 1, wherein the catalyst gas units are formed by oxidation catalysts.

4. The measuring device according to claim 1, wherein a reference gas flow with a hydrocarbon concentration in the upper measurement range of the two sensors can be fed to the two sensors.

5. The measuring device according to claim 1, wherein a filter member for filtrating the measuring gas flow is provided forward of the sensors in the flow direction.

6. The measuring device according to claim 1, wherein a drying element for drying the measuring gas flows is provided forward of the sensors in the flow direction.

7. The measuring device according to claim 5, wherein the drying element is configured as a membrane dryer.

8. A method for detecting the hydrocarbon content in a gas flow, comprising:
continuously feeding a first measuring gas flow to a first sensor configured as a metal oxide semiconductor gas sensor,
determining the hydrocarbon content in the first measuring gas flow and producing a first measurement result by means of the first sensor,
discontinuously feeding a second measuring gas flow to a second sensor configured as a photoionization sensor,
determining the hydrocarbon content in the second measuring gas flow and producing a second measurement result by means of the second sensor,
evaluating the measurement results of the two sensors,
producing a catalyst gas flow, wherein the catalyst gas flow is fed to the second sensor when the second sensor is turned off or not used.

9. The method according to claim 8, wherein
the second sensor is activated only immediately before use,
an automatic zero adjustment is carried out after a sufficient stabilization time,
the measuring gas flow is fed after the zero adjustment.

10. The method according to claim 8, wherein, subsequent to the parallel measurement of the two sensors, the measurement results of the two sensors are compared and the first sensor is calibrated if necessary.

11. The method according to claim 8, wherein the measuring gas flows are filtrated by means of a filter member prior to being fed to the two sensors.

12. The method according to claim 8, wherein the measuring gas flows are dried by means of a drying element prior to being fed to the two sensors.

13. The method according to claim 8, further comprising a calibration of the two sensors by means of a reference gas flow with a hydrocarbon concentration in the upper measurement range of the two sensors.

14. The method according to claim 8, further comprising a compensation of a cross sensitivity of the first sensor if the measured values of the second sensor are worse than Class 1 (ISO 8573), by
a. using a current measurement result of the second sensor as a reference quantity for a statistical probability calculation of previously determined calibration values of the first sensor,
b. removal of the most improbable calibration values from the collection of the previous calibration values,
c. determination of a slope of a measurement curve from a mean value resulting from the collection of the previous calibration values after the removal of the most improbable calibration values,
d. adjustment of a system slope by means of a filter.

15. The method according to claim 8, further comprising a correction of an offset value of the first sensor if the measured values of the second sensor are better than Class 1 (ISO 8573), by
a. determining an operating point on a measurement curve from the measurement results of the first sensor,
b. calculation of values for correcting the offset value of the first sensor,
c. taking into account the exponential characteristic curve of the first sensor in the determination of the offset value.
